(19) United States
(12) Patent Application Publication
(10) Pub. No.: US 2006/0122496 A1
(43) Pub. Date: Jun. 8, 2006

(54) Method, apparatus, and system for automatically positioning a probe or sensor

(76) Inventors: Mark George, Sullivan's Island, SC (US); Daryl Bohning, Warren, CT (US)

Correspondence Address: NEEDLE & ROSENBERG, P.C., SUITE 1000, 999 PEACHTREE STREET, ATLANTA, GA, 30309-3915, US

(21) Appl. No.: 10/991,129

(22) Filed: Nov. 17, 2004

(51) Int. Cl. A61B 5/05

(52) U.S. Cl. 600/424

(57) ABSTRACT

A self-contained hardware/software system has been designed to provide anatomy-referenced positioning of a probe or sensor, such as a TMS coil, with respect to a subject. The system may be used in interleaved TMS/fMRI studies of brain circuitry. The system allows the TMS coil to be positioned with millimeter accuracy based on anatomical target locations selected in an MR volume of a subject's brain acquired at the beginning of the interleaved TMS/fMRI study. The TMS coil may then be held in that position during subsequent scans.

### CROSS-REFERENCE TO RELATED APPLICATIONS

This application claims priority to U.S. Provisional Application No. 60/381,411 filed May 17, 2002, U.S. Provisional Application No. 60/427,802 filed Nov. 20, 2002. International Patent Application No. PCT/US03/15300 filed May 16, 2003. All of these applications are hereby incorporated by reference.

### BACKGROUND

The present invention relates generally to the positioning of a probe or sensor. More particularly, the present invention relates to the automatic positioning of a probe or sensor with respect to a subject using magnetic resonance imaging.

The combination of transcranial magnetic stimulation (TMS) with neuroimaging has potential in the studying of effective conductivity of brain circuits and has afforded new opportunities for investigation of cortical function. Development of techniques for neuronavigation based upon individual anatomic and functional images remains an area of concentrated investigation.

It is possible to interleave TMS with functional magnetic resonance imaging (fMRI) to visualize regional brain activity in response to direct, non-invasive stimulation. Details of this interleaving are described, for example, in a commonly assigned PCT Application entitled “Method, Apparatus, and System for Determining Effects and Optimizing Parameters of Vagus Never Stimulation”, filed on or about May 5, 2003, Attorney Docket No. 19113.0094P1 hereby incorporated by reference. A major practical difficulty in this effort is accurately positioning and holding the TMS coil for stimulation, and further, relating its position to brain anatomy.

Positioning a transcranial magnetic stimulation (TMS) coil on the scalp of a subject using a probabilistic approach based on average locations of cortical anatomy lacks both accuracy and precision for individual subjects. Analysis of cortical response to TMS based upon mapping of TMS location to separately obtained anatomic images cannot demonstrate direct temporal causation.

There are commercial sterotactic systems which use magnetic resonance (MR) images on a special workstation combined with a probe at the end of an articulated arm. The position of the probe is displayed on a display of the MR image. However, these systems are not MR compatible so they cannot work during an MR study. Also, these systems are not capable of holding a TMS coil and do not actually position the probe. They only show the probe's position relative to the MR images.

Efforts to modify a surgical robot so that a TMS coil can be mounted at the end of the robotic arm so that a TMS coil may be positioned over a point identified in an MR image displayed on a console have as yet been unsuccessful. Even if such efforts were successful, such a device would not be MR-compatible so it could not operate in real time in conjunction with an MR scanner. Thus, it would be far too complex and expensive for use in MR-guided positioning of a TMS coil during office visit TMS treatments.

### SUMMARY

According to an exemplary embodiment, a probe or sensor is positioned with respect to a subject by obtaining a magnetic resonance image of at least a portion of the subject, determining an optimal position for the probe or sensor with respect to the subject, based on the magnetic resonance image, and moving the probe or sensor to the optimal position.

In one embodiment, a coil is positioned for applying transcranial magnetic stimulation (TMS) to an optimal position with respect to the subject's brain. The TMS application may be interleaved with functional magnetic resonance imaging (fMRI). The positioning may be performed at the beginning of an interleaved TMS/fMRI study, and the TMS coil may be held in place through the remainder of the TMS/fMRI study.

In another embodiment, the TMS coil may be moved with respect to a subject's scalp until a particular motor response is observed, and the settings for the coil position may be entered into a processor. Then, based on these settings, a point on the scalp of the subject contacted by transcranial magnetic stimulation may be computed. Also, a point of maximum TMS magnetic field intensity may be computed. This may be used to determine a relation of the transcranial magnetic stimulation and effects on particular areas of the brain. This may be useful for applications to the cerebral cortex, in which the point of maximum TMS coil magnetic intensity is computed at the depth of the cerebral cortex. A relation between the TMS coil's field pattern to the subject's brain anatomy and the areas of the brain showing fMRI activation may be determined.

These and other aspects will become apparent from the following description of the preferred embodiment taken in conjunction with the following drawings, although variations and modifications may be effected without departing from the spirit and scope of the novel concepts of the disclosure.

### BRIEF DESCRIPTION OF THE DRAWINGS

FIG. 1 illustrates an exemplary device for positioning a probe/sensor;

FIG. 2 provides a more detailed schematic of an exemplary device for radial positioning of a support spar on which the probe/sensor is mounted;

FIGS. 3A and 3B provide an exemplary top view and side view, respectively, of the support spar;

FIGS. 4A and 4B illustrate an exemplary side view and front view, respectively, of a head positioning setup;

FIG. 5 illustrates an exemplary chair mounted device;

FIG. 6 shows an exemplary schematic of a TMS coil positioner and holder;

FIGS. 7 and 8 show an exemplary user interface;

FIG. 9 illustrates exemplary cycles of TMS application; and

FIGS. 10A-10C illustrate exemplary results of TMS application from a representative subject.

### DETAILED DESCRIPTION

According to exemplary embodiments, a new magnetic-resonance (MR) compatible device, system and method have been developed for flexibly, accurately and repeatably positioning a probe, e.g., a stimulator, or a sensor, over a person's head so as to be directly above a point in the brain identified in an MR image. The device, system, and method are adaptable to a variety of MR and PET scanners as well as a variety of floor and chair-mounted stands for office treatments or testing.

According to an exemplary embodiment, the device translates the coordinates of a point of interest in the brain, obtained from a standard set of MR images detailing the brain's 3D anatomy, into settings for the device so that it will position the probe over the point of interest. In one embodiment, this translation may be performed in real time, and positioning of the probe or sensor may be performed automatically and in real time.

The device may be constructed with multiple degrees of freedom and a consistent, mutually orthogonal, geometry to provide almost complete coverage of the cortex of the brain.

The transformation from the MR scanner coordinates to device settings uses a fast, accurate algorithm that can be installed on either a standalone computer or on the scanner's computer. No expensive additional workstation or expensive systems of articulate arms are required.

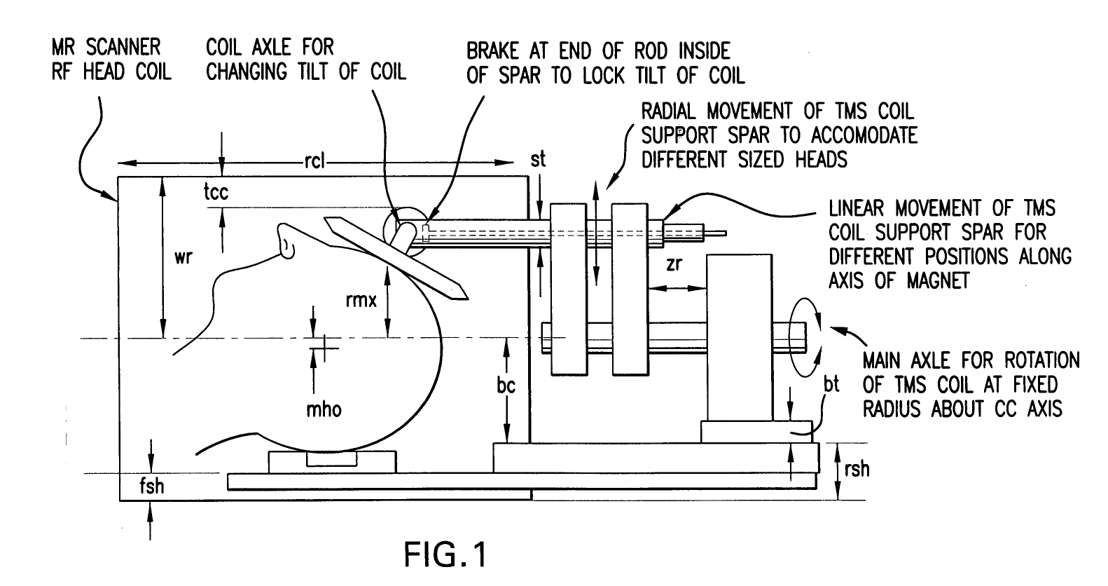

FIG. 1 shows an overview of an exemplary device, mounted in back of an MR scanner RF head coil.

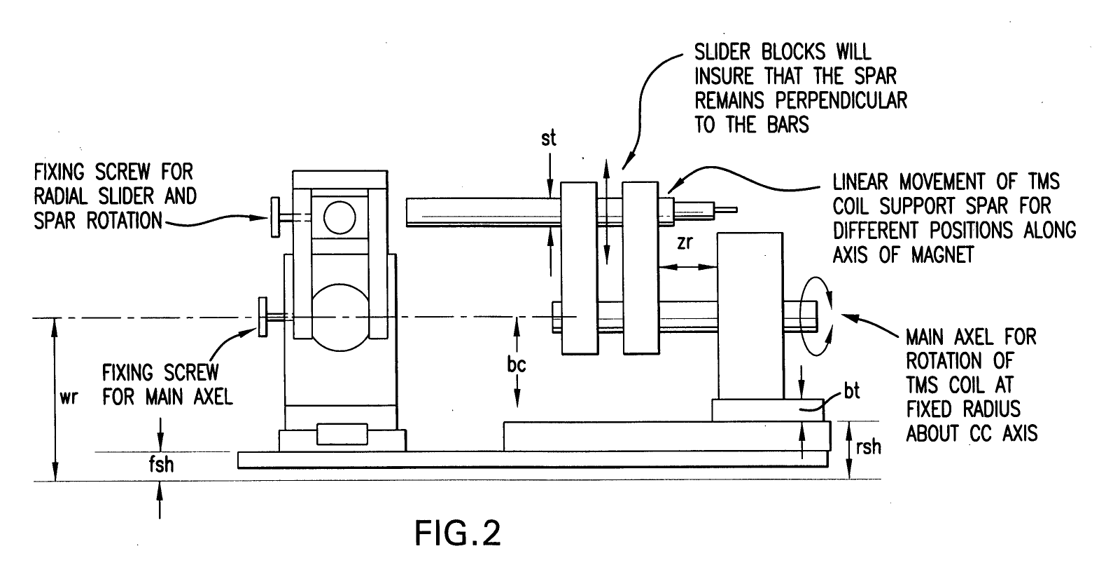

FIG. 2 provides a more detailed schematic of an exemplary device for radial positioning of a support spar on which the probe/sensor is mounted.

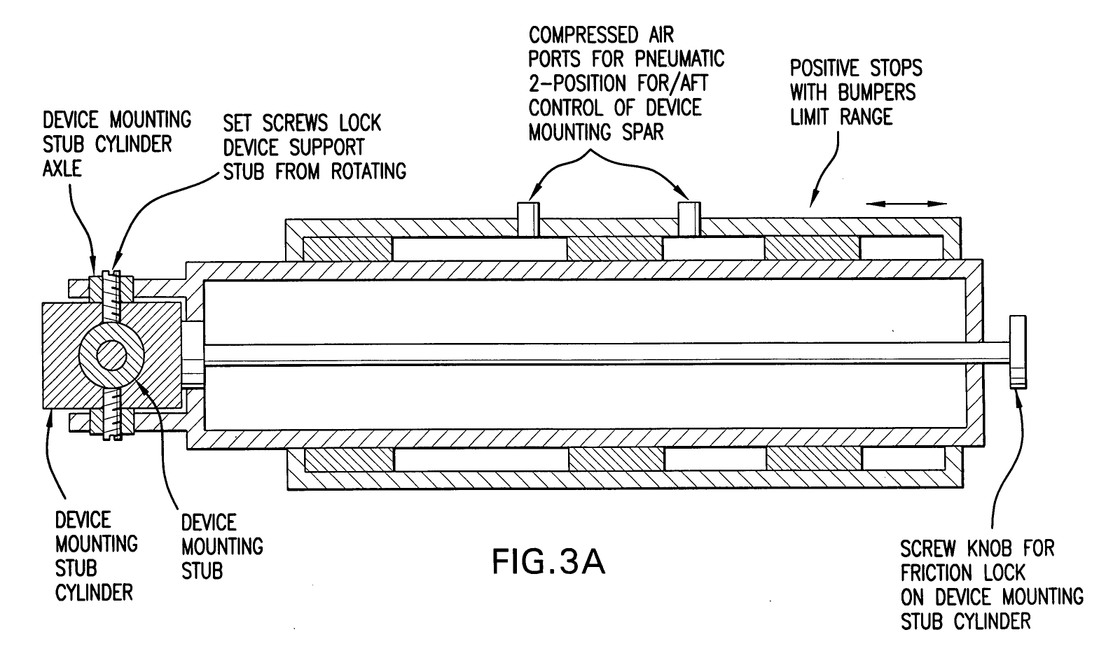

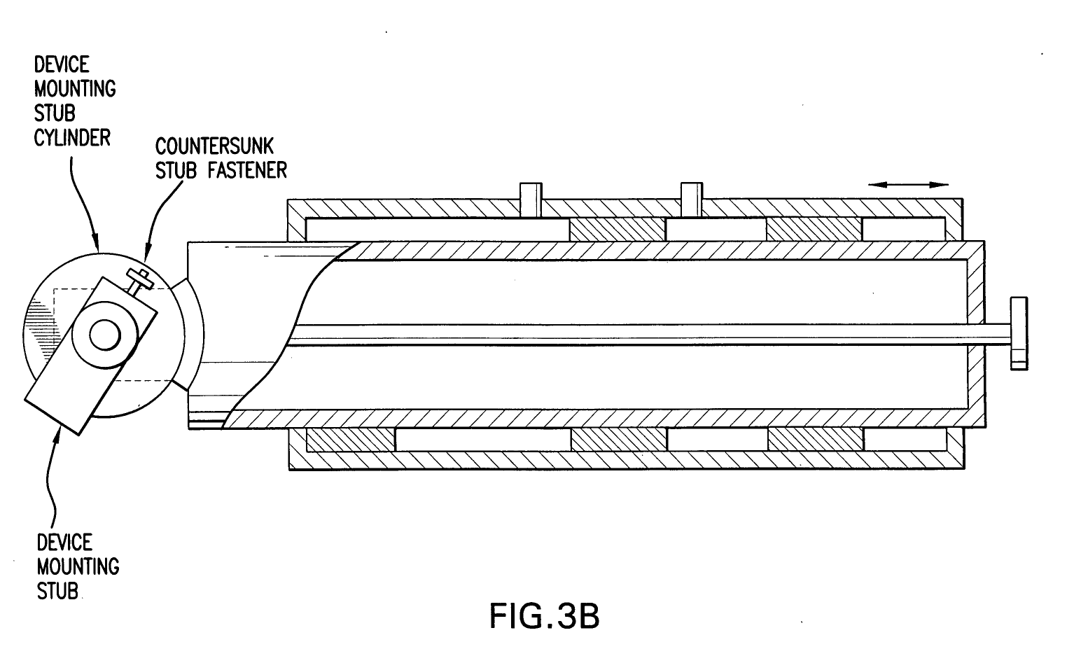

FIGS. 3A and 3B provide a top view and a side view, respectively, of the support spar. This drawing shows how the probe/sensor mounting stub is attached to the end of the spar and how the pneumatic fore/aft movement may be implemented.

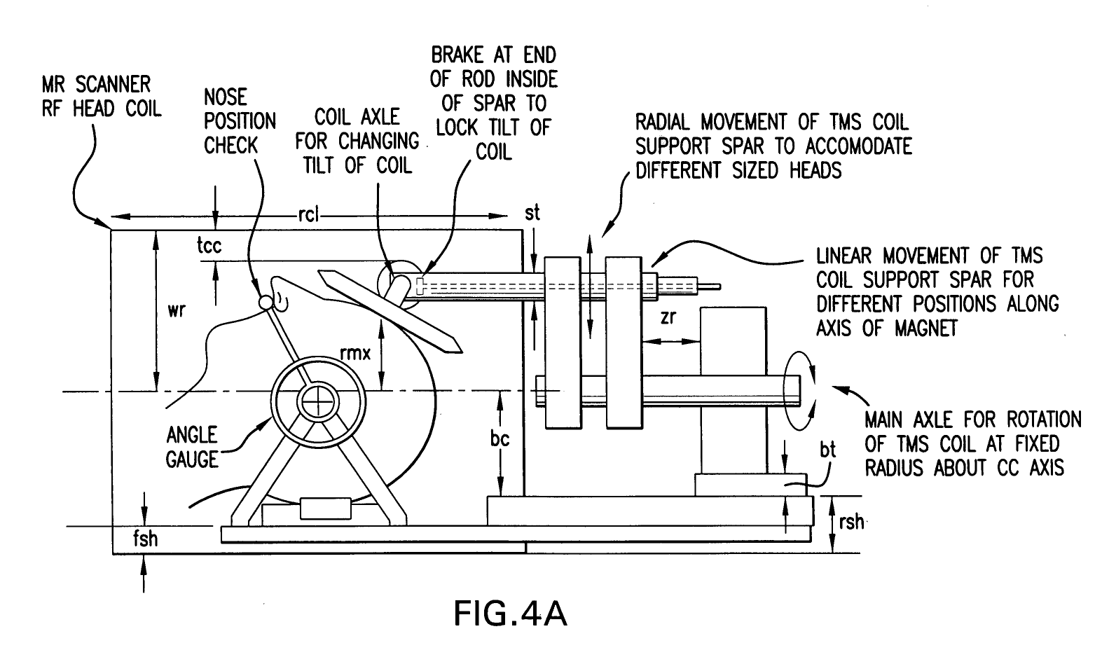

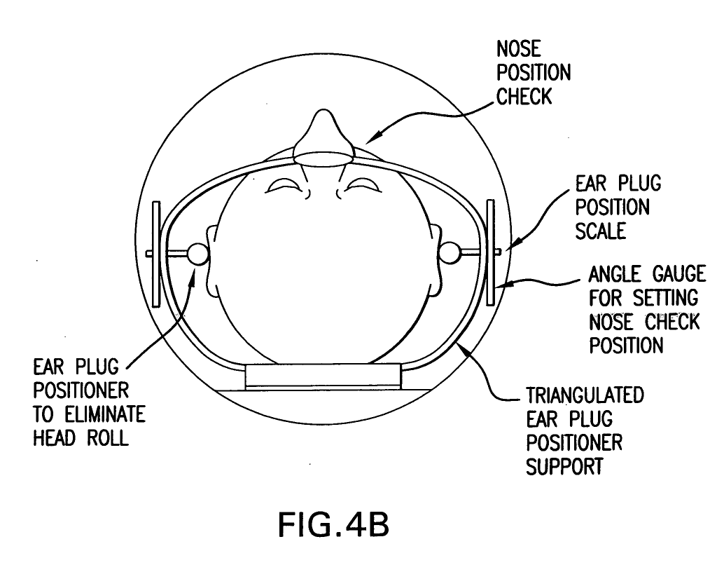

FIGS. 4A and 4B illustrate a side view and a front view, respectively, of an exemplary head positioning setup. Adjustable padded ear plugs eliminate head roll, and an under the nose check eliminates head pitch changes.

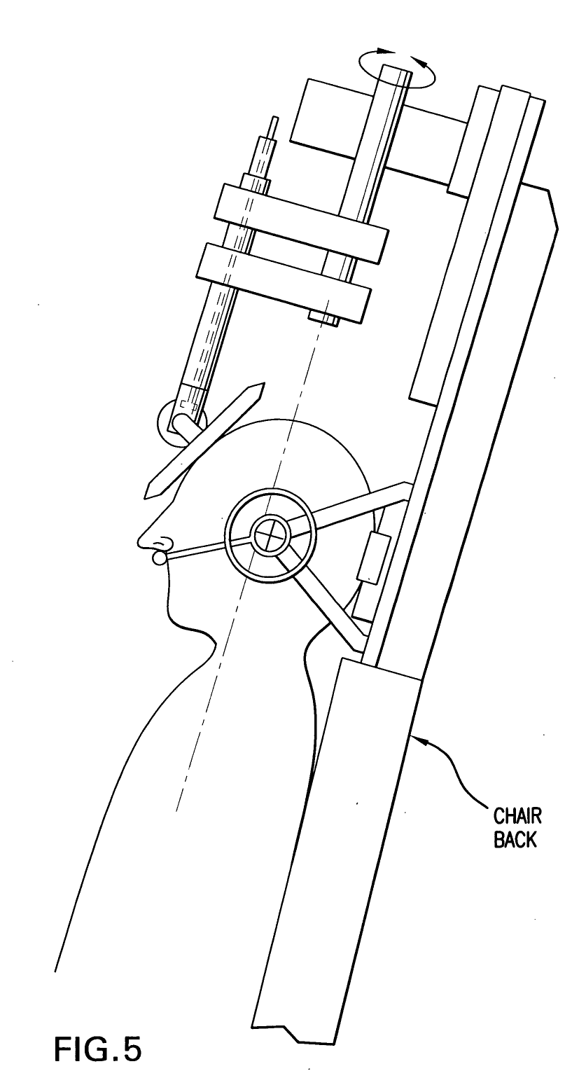

FIG. 5 illustrates an exemplary chair-mounted positioner.

According to an exemplary embodiment, the probe/sensor may be a coil for applying transcranial magnetic stimulation (TMS). The application of the TMS may be interleaved with functional magnetic resonance imaging (fMRI).

According to exemplary embodiment, a hardware/software system has been developed for positioning the TMS coil based on a target location selected in an MR volume acquired at the beginning of an interleaved TMS/fMRI study. According to one embodiment, the TMS coil may be positioned on the scalp so that the coil-field isocenter line is directed at a selected target on the subject's individual cortical anatomy. Then, the TMS coil is held securely in that position during the subsequent scans.

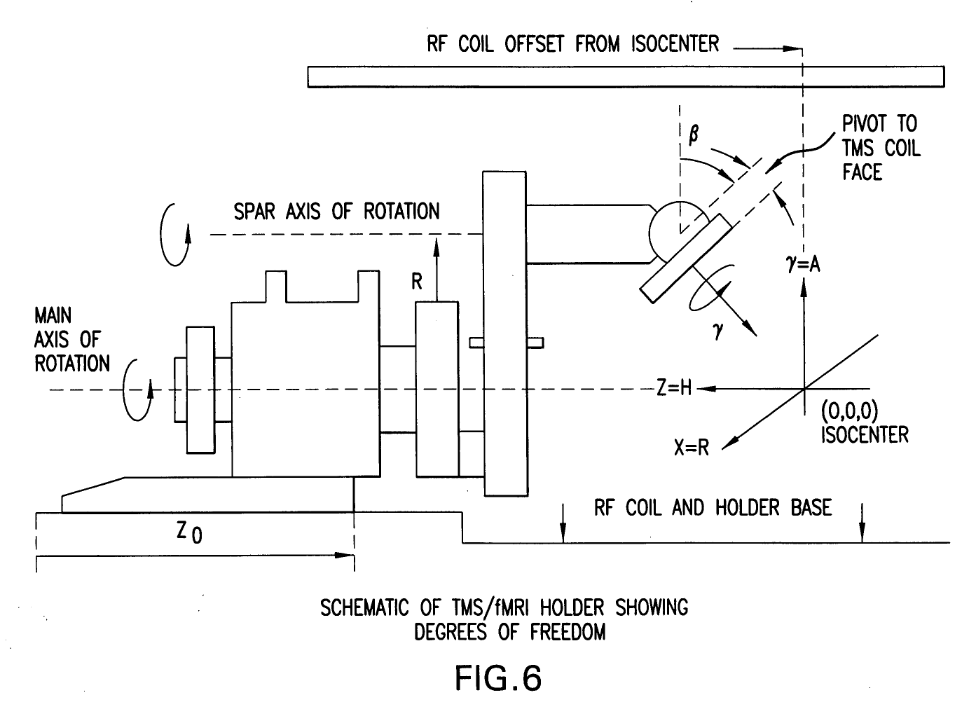

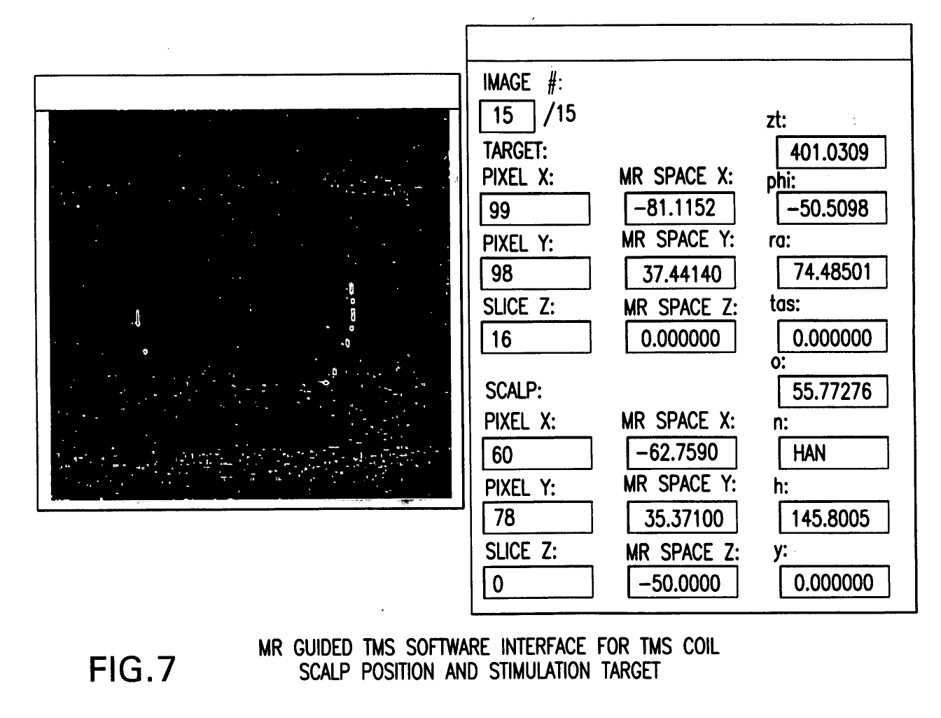

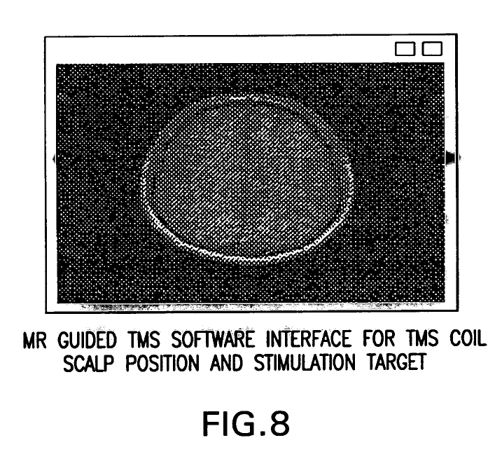

FIG. 6 shows a schematic of an exemplary TMS coil positioner and holder illustrating six (6) scaled degrees of freedom which allow the TMS coil to be moved to any point on the subject's scalp and then oriented so as to stimulate a selected target in the cerebral cortex. FIGS. 7 and 8 show the user interface which lets an investigator load an image volume and select the scalp placement and TMS simulation target positions. The software then computes the correct settings for the positioner/holder.

Those skilled in the art will appreciate that the user interface may be associated with a Macintosh operating system or other any other computer operating systems, such as PC, OS2, Unix, etc.

According to an exemplary embodiment, a subject first lies on a scanner bed and places his or her head in the head cradle of the device. The head is then centered and restrained with foam padding, and the subject is moved into the scanner. A high resolution structural MR is taken and loaded into the MRGuidedTMS software for selection of the scalp and target positions. The subject is then brought out of the scanner, and the TMS coil is positioned according to the settings computed by the software. Finally, the subject is put back into the scanner for the study.

Alternatively, in cases where the application is the motor cortex, and TMS stimulation site has been determined by moving the TMS coil until the associated motor response is observed, the investigator can enter the settings of the holder, and the software will compute the point of scalp contacted and the point of maximum TMS coil magnetic field intensity at the depth of cerebral cortex. This makes it possible to determine the relation of the TMS coil's field pattern to that individual's brain anatomy and the areas showing fMRI activation.

The holder also includes a facility for pneumatically shifting the TMS coil away from the subject's head to reduce the static susceptibility artifact it causes, as a precaution. This is an optional feature for uses at field strengths of roughly 1.5 T. This feature becomes more relevant and necessary at higher field strengths (3-4 T).

To illustrate exemplary results of the system, method, and device, a series of calibration scans were made with the TMS coil replaced by a probe with two MR visible point sources at 5 cm and 12 cm, respectively, from the holder pivot (β-angle). In the prototype positioner/holder, TMS targeting is performed with an accuracy of dx=±6.2 mm, dz=±4.7 mm. Accuracy is expected to improve in production devices due to reduction in manufacturing tolerance and a built-in reference to eliminate MR scanner bed reference and position errors, which are the major cause of the error in the z-direction.

In an ongoing study, to date, four healthy adult volunteers (mean age 39 yr. SD 18, 2 women, 1 left-handed man) gave informed consent in accord with procedures approved by the Institutional Review Board and were scanned up to three times each. One subject did not complete all scans due to claustrophobia and so only provided motion-elicitation data and not BOLD imaging data. The subjects' heads rested on a stiff foam support and were stabilized with foam-padded Velcro straps. Permanent marks on molded earplugs were aligned with plastic rods on an adjustable frame mounted to the receiving coil base. Adjustment at the initial scan determined a comfortable position. For subsequent scans, heads were re-aligned to rods connected to the earplugs. Vision was unconstrained.

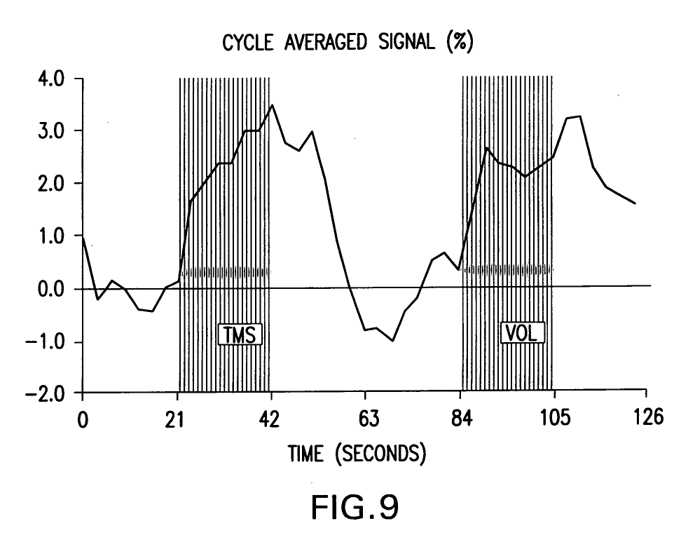

A Dantec MagPro® stimulator with a non-ferromagnetic figure-8 coil and 8 m cable (Dantec Medical A/S, Skovlunde, Denmark) provided TMS. The TMS coil was held by a head-coil mounted apparatus that could be adjusted and fixed to hold the coil rigidly. Scanning was performed on a Picker EDGE 1.5 T scanner. A cortical target, on the lateral aspect of the hand knob (approx x37, y-23, z59 in Talairach) was selected from an initial transverse T1 weighted scan on each individual subject. The spatial location of the selected voxel relative to the scanner isocenter was recorded from the interface software. Subsequent sagittal and oblique coronal scans were centered on the target location. The coronal scan was angled to be perpendicular to the AP curve of the scalp as shown in the sagittal scan. The oblique coronal image through the target point was used to establish the scalp location which would allow the isocenter line of the TMS coil to intersect the anatomical target. The six coordinates of these points were used to calculate the required settings on the TMS coil holder to allow locking the coil in the appropriate location and orientation against the scalp. Once the coil was in position, interleaved TMS-fMRI was performed to observe the elicited BOLD response. TMS at 110% of motor threshold (MT=level inducing movement on 50% of pulses) caused consistent movement. Functional scans used a gradient echo, single-shot, echo-planar fMRI sequence (tip angle=90%, TE=40 ms, TR=3.0 s, FOV=27.0 cm, matrix=128×128, 15 6 mm axial slices, 1 mm gap, frequency selective fat suppression). Scans (15.2 min) lasted for 7 cycles of 6, 21-second epochs each: Rest-TMS-Rest-Rest-VOL-Rest. “Rest”=no task, “TMS”=TMS stimulation at 110% MT, “VOL”—volitional mimic of TMS-induced movement, cued by low level (20% MT) pulses. (See FIG. 9). During task epochs, TMS pulses occurred after every fifth image (1 Hz) in trains of 21 pulses.

Data were processed on Sun SPARCstations (Sun Microsystems, Mountain View, Calif.) using SPM99 (Wellcome Dept. Cognitive Neurol., London UK). Image sets were realigned to the first volume acquired. Statistical parametric maps, SPM(t)'s, were calculated for condition specific (TMS or VOL) effects within a general linear model. Modeled epochs were convolved with a canonical hemodynamic response function. Estimated movement parameters (six) were used as confounds in the linear model design matrix. Temporal high-pass filtering was carried out with cutoff frequency at twice the cycle length (252 s). Thresholding of the t-maps was carried out at a p=0.10 corrected for multiple comparisons. All clusters examined had p values less than 0.05 when assessed by spatial extent.

Seven trials with four subjects were performed. In all cases, motion of the thumb (chiefly abduction) was produced when the TMS was positioned based on the anatomic image using the above system, with TMS intensity levels within 5% of individual threshold levels determined six to twelve months previously. The brain imaging results revealed that in all cases, BOLD response clusters were observed within four mm of the selected hand knob target.

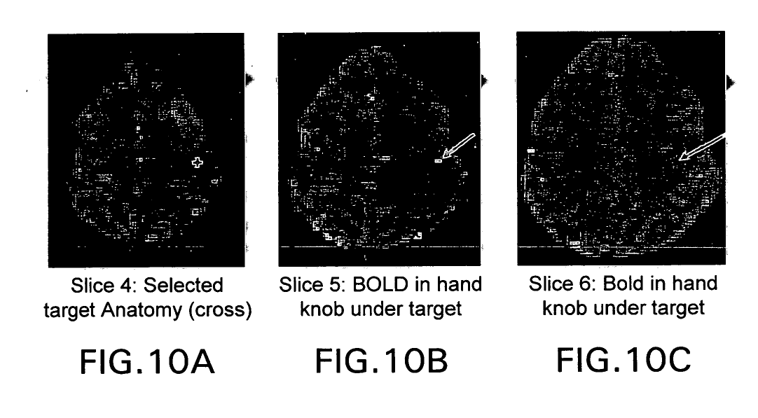

FIGS. 10A-10C shows results from a representative subject. The white cross on slice 4 indicates the voxel chosen as the target. BOLD response was observed directly below the chosen target location (arrows, slices 5 and 6). This pattern was true of all runs that has usable BOLD data (6 of 7 scans). Time-intensity curves from hand knob clusters displayed peaks during task epochs of 2-4% of the cluster mean intensity.

This work was funded in part by an NINDS grant (ROI RR14080-02).

These initial results demonstrate that this system can produce accurate and precise positioning of TMS stimulation based on individual brain anatomy for use in interleaved TMS-fMRI studies. Such an approach will allow analysis of the mechanisms of TMS-evoked BOLD response of the cortex at previously unattainable levels of temporal and spatial resolution. The hardware/software system allows MR-guided TMS coil positioning for interleaved TMS/fMRI studies with millimeter accuracy. Positioning accuracy depends on holder scale reading, holder tolerances, and MR scanner bed referencing and positioning.

The present design is simple to use, sufficiently accurate for both research and clinical treatment, and inexpensive enough for any TMS practitioner to afford.

While there have been shown preferred and alternate embodiments of the present invention, it is to be understood that certain changes can be made in the form and arrangement of the elements of the system and steps of the method as would be know to one skilled in the art without departing from the underlying scope of the invention as described herein. Furthermore, the embodiments described above are only intended to illustrate the principles of the present invention and are not intended to limit the scope of the invention.

What is claimed is:

1. A method for positioning a probe or sensor with respect to a subject, comprising the steps of:
obtaining a magnetic resonance image of at least a portion of the subject;
determining an optimal position for the probe or sensor with respect to the subject, based on the magnetic resonance image; and
moving the probe or sensor to the optimal position.

2. The method of claim 1, wherein the steps are performed for moving a coil for applying transcranial magnetic stimulation (TMS) to an optimal position with respect to the subject's brain.

3. The method of claim 2, wherein the transcranial magnetic stimulation is interleaved with functional magnetic resonance imaging (fMRI).

4. The method of claim 3, wherein the steps of obtaining, determining, and moving are performed at the beginning of an interleaved TMS/fMRI study.

5. The method of claim 4, wherein the TMS coil is held in place through the remainder of the TMS/fMRI study.

6. A method for determining effects of transcranial magnetic stimulation (TMS) on a subject's brain, comprising the steps of:
computing a point on the scalp of the subject contacted by transcranial magnetic stimulation and computing a point of maximum TMS magnetic field intensity based predetermined settings of a coil position, wherein the computed points are used to determine a relation of the transcranial magnetic stimulation and effects on particular areas of the brain.

7. The method of claim 6, wherein the settings for the coil position are predetermined by moving a coil supplying transcranial magnetic stimulation with respect to a subject's scalp until a particular motor response is observed and entering the settings for the coil position.

8. The method of claim 6, wherein the TMS is applied to the cerebral cortex, and the step of computing includes computing the point of maximum TMS coil magnetic intensity at the depth of the cerebral cortex.

9. The method of claim 6, wherein the application of the TMS is interleaved with functional magnetic resonance imaging (fMRI), and the step of determining determines a relation between the TMS coil's field pattern to the subject's brain anatomy and the areas of the brain showing fMRI activation.

10. A device for automatically positioning a probe or sensor with respect to a subject, comprising the steps of:
means for obtaining a magnetic resonance image of at least a portion of the subject;
means for determining an optimal position for the probe or sensor with respect to the subject, based on the magnetic resonance image; and
means for automatically moving the probe or sensor to the optimal position.

11. The device of claim 10, wherein the probe or sensor includes a coil for applying transcranial magnetic stimulation (TMS) to an optimal position with respect to a subject's rain.

12. The device of claim 11, wherein the transcranial magnetic stimulation is interleaved with functional magnetic resonance imaging (fMRI).

13. The device of claim 12, wherein the TMS coil is moved to the optimal position at the beginning of an interleaved TMS/fMRI study.

14. The device of claim 13, wherein the TMS coil is held in place through the remainder of the TMS/fMRI study.

15. A device for determining effects of transcranial magnetic stimulation (TMS) on a subject's brain, comprising:
means for computing a point of the scalp contacted by transcranial magnetic stimulation from a coil and means for computing a point of maximum TMS magnetic field intensity based on predetermined settings of a coil position, wherein the computed points are used for determining a relation of the transcranial magnetic stimulation and effects on particular areas of the brain.

16. The device of claim 15, wherein the settings for the coil position are predetermined by moving a coil supplying transcranial magnetic stimulation with respect to a subject's scalp until a particular motor response is observed and entering settings for the coil position.

17. The device of claim 15, wherein the TMS is applied to the cerebral cortex, and the means for computing the point of maximum TMS coil magnetic intensity computes the intensity at the depth of the cerebral cortex.

18. The device of claim 15, wherein the application of the TMS is interleaved with functional magnetic resonance imaging (fMRI), and the step of determining determines a relation between the TMS coil's field pattern to the subject's brain anatomy and the areas of the brain showing fMRI activation.

19. A system for automatically positioning a probe or sensor with respect to a subject, the system comprising:
a magnetic resonance imaging device for providing a magnetic resonance image of at least a portion of the subject;
a processor for determining an optimal position for the probe or sensor with respect to the subject, based on the magnetic resonance image; and
a movable arm for automatically moving the probe or sensor to the optimal position.

20. The system of claim 19, wherein the probe or sensor includes a coil for applying transcranial magnetic stimulation (IMS) to an optimal position with respect to the subject's brain.

21. The system of claim 20, wherein the transcranial magnetic stimulation is interleaved with functional magnetic resonance imaging (fMRI).

22. The system of claim 21, wherein the coil is moved to the optimal position at the beginning of an interleaved TMS/fMRI study.

23. The system of claim 22, wherein the TMS coil is held in place through the remainder of the TMS/fMRI study.

24. A system for determining effects of transcranial magnetic stimulation (TMS) on a subject's brain, comprising:
a positioner for positioning a TMS coil supplying the transcranial magnetic stimulation based on predetermined settings; and
a processor for computing a point of the scalp contacted by transcranial magnetic stimulation from a coil and a point of maximum TMS magnetic field intensity based on the predetermined settings of the coil position, wherein the computed points are used to determine a relation of the transcranial magnetic stimulation and effects on particular areas of the brain.

25. The system of claim 24, wherein the settings are predetermined by the positioner moving the coil supplying transcranial magnetic stimulation with respect to a subject's scalp until a particular motor response is observed, and entering those settings representing the coil position into the processor.

26. The system of claim 24, wherein the TMS is applied to the cerebral cortex, and the step of computing includes computing the point of maximum TMS coil magnetic intensity at the depth of the cerebral cortex.

27. The system of claim 24, wherein the application of the TMS is interleaved with functional magnetic resonance imaging (fMRI), and the step of determining determines a relation between the TMS coil's field pattern to the subject's brain anatomy and the areas of the brain showing fMRI activation.
